(19) United States
(12) Patent Application Publication
(10) Pub. No.: US 2026/0241152 A1
(43) Pub. Date: Aug. 20, 2026

(54) WOUND CARE DRESSING PAD

(71) Applicant: CaseMed Engineering, LLC, Waukegan, IL (US)

(72) Inventor: Casey Samangooie, Wadsworth, IL (US)

(21) Appl. No.: 19/530,628

(22) Filed: Feb. 5, 2026

Related U.S. Application Data
(60) Provisional application No. 63/759,697, filed on Feb. 18, 2025.

(51) Int. Cl. A61M 35/00; A61F 13/00; A61F 13/02

(52) U.S. Cl. CPC A61M 35/10; A61F 13/00063; A61F 13/0266; A61F 2013/00285; A61M 2202/064; A61M 2205/0238

(57) ABSTRACT

A wound care dressing pad includes a first film layer, a first impermeable film layer formed to include at least one reservoir, a second impermeable film layer coupled to the first impermeable film layer to enclose the at least one reservoir, an impregnable substrate layer coupled to the second impermeable layer, and a peel-off backing layer removably coupled to the first film layer. The first impermeable film layer is coupled to the first film layer such that the at least one reservoir extends through the first film layer. The first film layer extends beyond a periphery of the first impermeable layer. The second impermeable film layer has an opening disposed therethrough and aligned with each of the at least one reservoirs of the first impermeable film layer. The impregnable substrate layer is coupled to the second impermeable layer around at least a periphery of the impregnable substrate layer.

### CROSS-REFERENCE TO RELATED APPLICATIONS

This application claims the benefit of and priority to U.S. Provisional Application No. 63/759,697, filed February 18, 2025, which is hereby incorporated by reference herein in its entirety.

### FIELD OF THE INVENTION

The present invention relates generally to a wound care dressing pad, and in particular to a wound care dressing pad having one or more liquid reservoirs for delivery of a liquid, gel, cream, or powder medicament to a wound.

### BACKGROUND OF THE INVENTION

Bandages can be applied over wounds to protect the wound from the environment and promote healing. Medicament creams and ointments can be applied to the wound before application of the bandage. However, a need exists for wound care dressing pad having one or more liquid reservoirs for delivery of a liquid, gel, cream, or powder medicament to a wound. The present disclosure provides a solution to these and other needs.

### SUMMARY OF THE INVENTION

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

According to certain aspects of the present disclosure, a wound care dressing pad includes a first film layer, a first impermeable film layer formed to include at least one reservoir, a second impermeable film layer coupled to the first impermeable film layer around at least a periphery of the at least one reservoir to enclose the at least one reservoir, an impregnable substrate layer coupled to the second impermeable layer, and a peel-off backing layer removably coupled to the first film layer. The first impermeable film layer is coupled to the first film layer such that the at least one reservoir extends through the first film layer. The first film layer extends beyond a periphery of the first impermeable layer. The second impermeable film layer has an opening disposed therethrough and aligned with each of the at least one reservoirs of the first impermeable film layer. The impregnable substrate layer is coupled to the second impermeable layer around at least a periphery of the impregnable substrate layer.

According to certain aspects of the present disclosure, the second impermeable film layer has one opening disposed therethrough. A pull tab is removably coupled to the second impermeable film layer to cover the one opening. A portion of the pull tab extends beyond the first film layer.

According to certain aspects of the present disclosure, the first impermeable film layer is formed to include two reservoirs. The second impermeable film layer has two openings disposed therethrough, and each of the openings is aligned with one of the two reservoirs. A pull tab is removably coupled to the second impermeable film layer to cover each of the two openings. A portion of each of the pull tabs extends beyond the first film layer.

According to certain aspects of the present disclosure, each of the two reservoirs contains a different medicament.

According to certain aspects of the present disclosure, at least one of the medicaments is a powder.

According to certain aspects of the present disclosure, the first impermeable film layer is formed to include two reservoirs. The second impermeable film layer has two openings disposed therethrough, each of the openings aligned with one of the two reservoirs. A single pull tab is removably coupled to the second impermeable film layer to cover both of the two openings, wherein a portion of the single pull tab extends beyond the first film layer.

According to certain aspects of the present disclosure, each of the two reservoirs contains a different medicament.

According to certain aspects of the present disclosure, at least one of the medicaments is a powder.

According to certain aspects of the present disclosure, the peel-off backing layer a printable label.

According to certain aspects of the present disclosure, the second impermeable film layer is covered with a hydrophilic coating.

According to certain aspects of the present disclosure, the first impermeable film layer and the second impermeable film layer each have a thickness in a range between about 2 mil and about 12 mil.

According to certain aspects of the present disclosure, a wound care dressing pad includes a first film layer, a first impermeable film layer formed to include at least one reservoir, a second impermeable film layer coupled to the first impermeable film layer around at least a periphery of the at least one reservoir to enclose the at least one reservoir, an impregnable substrate layer coupled to the second impermeable layer, and a third impermeable film layer coupled to the impregnable substrate layer. The first impermeable layer is coupled to the first film layer such that the at least one reservoir extends through the first film layer. The first film layer extends beyond a periphery of the first impermeable layer. The second impermeable film layer is coupled to the first impermeable film layer around at least a periphery of the at least one reservoir to enclose the at least one reservoir. The second impermeable film layer has a frangible opening aligned with each of the at least one reservoirs of the first impermeable film layer. The impregnable substrate layer is coupled to the second impermeable layer around at least a periphery of the impregnable substrate layer. The third impermeable film layer is coupled to the impregnable substrate layer around at least a periphery of the impregnable substrate layer.

According to certain aspects of the present disclosure, the first impermeable film layer is formed to include two reservoirs. The second impermeable film layer has two frangible openings disposed therethrough, each of the frangible openings aligned with one of the two reservoirs.

According to certain aspects of the present disclosure, each of the two reservoirs contains a different medicament.

According to certain aspects of the present disclosure, at least one of the medicaments is a powder.

According to certain aspects of the present disclosure, the third impermeable film layer is a printable label.

According to certain aspects of the present disclosure, the first impermeable film layer and the second impermeable film layer each have a thickness in a range between about 2 mil and about 12 mil.

According to certain aspects of the present disclosure, the frangible opening aligned with each of the at least one reservoirs of the first impermeable film layer is a line of weakness.

According to certain aspects of the present disclosure, the frangible opening aligned with each of the at least one reservoirs of the first impermeable film layer is a partial perforation arranged as a line.

According to certain aspects of the present disclosure, the frangible opening aligned with each of the at least one reservoirs of the first impermeable film layer is a partial perforation arranged as a circle.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure, and its advantages and drawings, will be better understood from the following description of representative embodiments together with reference to the accompanying drawings. These drawings depict only representative embodiments and are therefore not to be considered as limitations on the scope of the various embodiments or claims.

FIG. 1 shows an exemplary wound care dressing pad as seen from a wound application side on the left, and from a side facing away from a wound to be dressed on the right, according to certain aspects of the present disclosure.

FIG. 2 shows a cross-sectional schematic view taken along the lines 2-2 of FIG. 1, according to certain aspects of the present disclosure.

FIG. 3 shows a schematic view of the structure of an exemplary pull tab, according to certain aspects of the present disclosure

FIG. 4 shows an exemplary wound care dressing pad as seen from a side facing away from a wound to be dressed and having two pull tabs, according to certain aspects of the present disclosure.

FIG. 5 shows a cross-sectional schematic view taken along the lines 5-5 of FIG. 4, according to certain aspects of the present disclosure.

FIG. 6 shows a cross-sectional schematic view of another exemplary wound care dressing pad, according to certain aspects of the present disclosure.

FIG. 7 shows a cross-sectional schematic view of a further exemplary wound care dressing pad, according to certain aspects of the present disclosure.

FIG. 8 shows several examples of types of frangible openings, according to certain aspects of the present disclosure.

FIG. 9 shows a cross-sectional schematic view of an exemplary wound care dressing pad including liquid contents in the reservoir, according to certain aspects of the present disclosure.

FIG. 10 shows a cross-sectional schematic view of an exemplary wound care dressing pad including two reservoirs having a mix of contents in the reservoirs, according to certain aspects of the present disclosure.

### DETAILED DESCRIPTION

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

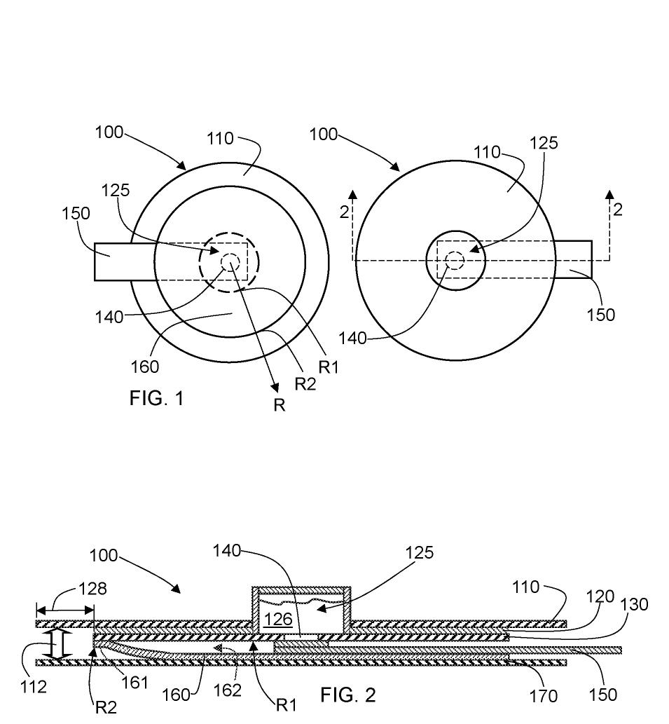

FIG. 1 illustrates an exemplary wound care dressing pad 100 as seen from a wound application side on the left in FIG. 1, and from a side facing away from a wound to be dressed on the right in FIG. 1. FIG. 2 shows a cross-sectional view taken generally along the lines 2-2 of FIG. 1. Referring generally to FIGS. 1 and 2, in an embodiment the wound care dressing pad 100 comprises a first film layer 110.

A first impermeable film layer 120 is coupled to the first film layer 110. The first impermeable film layer 120 is formed to include a reservoir 125. In some embodiments, the reservoir 125 is formed by pressing the material of the impermeable first layer 120 to bend and form the reservoir 125. In other embodiments, the reservoir 125 is formed form the impermeable film layer 120 as is known in the art.

The reservoir 125 is configured to contain, hold, or store a liquid, a gel, a cream, a powder contents 126 (See FIGS. 2, 9, and 10) or combinations thereof. In an embodiment the contents 126 of the reservoir 125 is a medicament or other material that is conducive to healing. For example, in an embodiment, the reservoir 125 holds an anti-biotic cream contents 126. In an embodiment, the reservoir 125 extends through the first film layer 110. In other embodiments, the first film layer 110 covers the reservoir 125 so that it does not protrude through the first film layer 110. The first film layer 110 extends beyond a periphery of the first impermeable film layer 120 by a distance, 128, thereby defining an annulus. In practice, the wound application side of the annulus includes an adhesive for securing the first film layer 110 to skin around a wound. As indicated by the arrow 112 in FIG. 2 (and FIG. 7), the wound application side of the annulus of the first layer 110 is initially covered by the peel-off backing layer 170, but upon removal of the peel-off backing layer 170, affixes to the skin around a wound. In an embodiment, the peel-off backing layer 170 is a printable label having contents or other information on it.

Referring to FIG. 2, a second impermeable film layer 130 is coupled to the first impermeable film layer 120 around at least a periphery of the reservoir 125 to enclose the reservoir 125. In practice, the second impermeable film layer 130 is permanently affixed to the first impermeable film layer 120 along a continuous path extending entirely around the reservoir 125. The continuous path of fixation is a ring of fixation in some embodiments, whereas in other embodiments a larger portion of the surface of the second impermeable film layer 130, including and extending radially beyond the ring of fixation, is permanently affixed to the first impermeable film layer 120.

For example, referring generally to FIGS. 1 and 2, in FIG. 1 a radial line, R, is provided on FIG. 1 showing radii R1 and R2 for reference. In FIG. 2, the second impermeable film layer 130 is permanently affixed to the first impermeable film layer 120 to form a complete seal around the reservoir 125 between and inclusive of any radius between R1 and R2. In some embodiments, there may be more than one ring of affixation in the range of radii R1 to R2, where the rings of affixation are separated by one or more radial spaces. The permanent path of fixation seals the reservoir 125.

In an embodiment, the second impermeable layer 130 has one or more openings or ports 140 or one or more frangible or perforated openings 240 aligned with the reservoir 125 of the first impermeable film layer 120. Hereinafter, an opening refers to an opening, a hole, a port, an aperture disposed through a layer of material. Also hereinafter, a frangible opening refers to a frangible, perforated, thinned, or otherwise weakened material that is not an open passage in an initial state but that which can be burst or broken open by application of pressure or force.

The one or more openings or one or more frangible openings are described more fully below in regard to FIGS. 4-8. Returning to FIG. 2 (and as also illustrated in FIG. 1), in an embodiment the second impermeable layer 130 has one opening 140 disposed therethrough. In an embodiment, a pull tab 150 is removably coupled to the second impermeable layer 130 to cover the one opening 140. To provide convenient access to a user, a portion of the pull tab 150 extends beyond the first film layer 110.

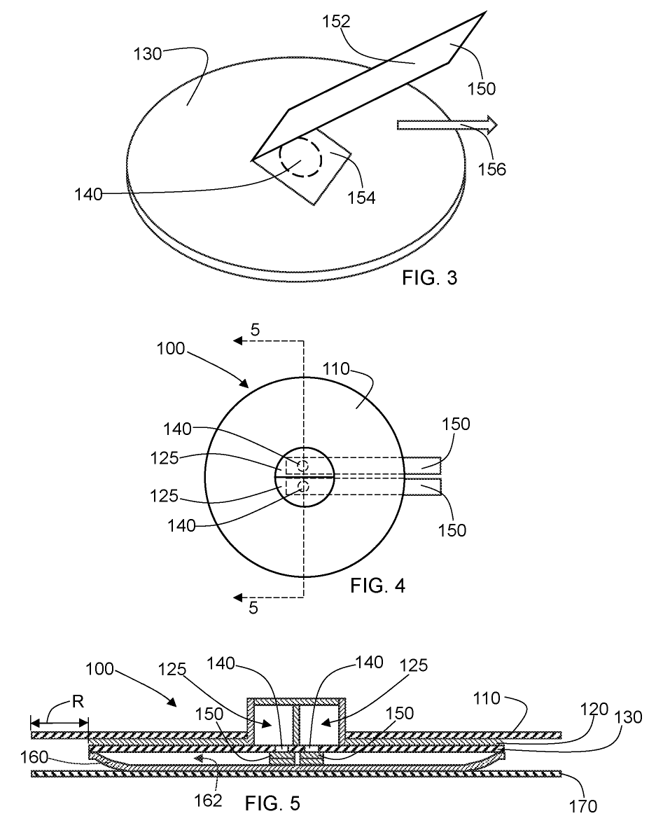

The pull tab 150 is removably coupled to the second impermeable layer 130, for example, by a releasable adhesive. Further, the pull tab 150 is designed to release from the second impermeable layer 130 without stretching, damaging, tearing, or otherwise deforming the second impermeable layer 130 or the one opening 140 disposed through the second impermeable layer 130. Referring to FIG. 3, in an embodiment the pull tab 150 includes a first portion 152 that is not directly affixed to the second impermeable layer 130. The first portion 152 folds back over a second portion 154 that is releasably affixed to over the one opening 140 to the second impermeable layer 130. The pull tab 150 is designed so that pulling it in the direction of the arrow 156 to release it causes the second portion 154 to gently peel off of the second impermeable layer 130 without stretching, damaging, tearing, or otherwise deforming the second impermeable layer 130 or the one opening 140 disposed through the second impermeable layer 130.

Returning now to FIGS. 1 and 2, in an embodiment, an impregnable substrate layer 160 is coupled to the second impermeable layer 130 around at least a periphery of the impregnable substrate layer 160. Except for a small gap where the pull tab 150 passes through, there is therefore a continuous region of fixation 161 around the impregnable substrate layer 160. This region of fixation 161 creates a pocket 162 between the impregnable substrate layer 160 and the second impermeable layer 130. For example, referring to FIG. 2, the impregnable substrate layer 160 is permanently affixed to second impermeable layer 130 proximate to the radius R2 to form a pocket having a radius that is less than the radius of the region of fixation 161.

When the pull tab 150 is pulled to uncover the one opening 140, the contents 126 of the reservoir 125 can flow into the pocket 162 and disburse laterally along the impregnable substrate layer 160, which helps in evenly being absorbed into and through the impregnable substrate layer 160. A peel-off backing layer 170 is removably coupled to the first film layer 110 to cover and protect the impregnable substrate layer 160 until the wound care dressing pad 100 is ready for use.

Referring now to FIGS. 4 and 5, in other embodiments, the wound care dressing pad 100 has a second impermeable layer 130 that has two openings 140 disposed therethrough. In this embodiment the reservoir 125 is actually two reservoirs 125. Each reservoir 125 is accessible by an opening 140 disposed through the second impermeable layer 130. Each reservoir 125 can contain the same or different contents 126 as described above and below. A pull tab 150 is coupled to the second impermeable layer 130 to cover each of the two openings 140. In some embodiments, a single pull tab 150 covers the two openings 140. In embodiments having three or more openings 140, a single pull tab 150 can cover all three or more of the openings 140, or a first pull tab 150 can cover a first one or more of the openings 140, a second pull tab 150 can cover a second one or more of the openings 140, etc., so that all of the openings are covered by a pull tab 150. A portion of each of the pull tabs 150 extends beyond first film layer 110.

When either or both of the pull tabs 150 is pulled to uncover its corresponding opening 140, or when the single pull tab 150 is pulled to uncover multiple of the one or more openings 140, the contents 126 of the reservoirs 125 can flow into the pocket 162 and disburse laterally along the impregnable substrate layer 160. This disbursement not only helps the contents 126 to evenly being absorbed into and through the impregnable substrate layer 160, but also, in the case of different contents 126 in each of the reservoirs 125, can further help in the mixing of the contents 126.

It should be noted that in other embodiments the number of reservoirs 125 can also be three, four, five, or more depending on the desired use of the wound care dressing pad 100. Regardless of the number of reservoirs 125, each reservoir 125 can contain the same or different contents 126 as described above and below. The geometry of the multiple reservoirs 125 can be spaced circumferentially around the wound care dressing pad 100 such that tabs 150 covering the openings 140 into the reservoirs 125 extend at circumferentially spaced angles relative to one another. For example, for three reservoirs 125, three tabs 150 can extend with a separation of 120 degrees between any two of the tabs 150. For four reservoirs 125, the spacing can be 90 degrees, and so forth. In other embodiments, the tabs 150 can be arranged in other patterns as desired to conveniently provide access to the openings 140 that are covered. As noted above, a single pull tab 150 can also cover two or more of the openings 140.

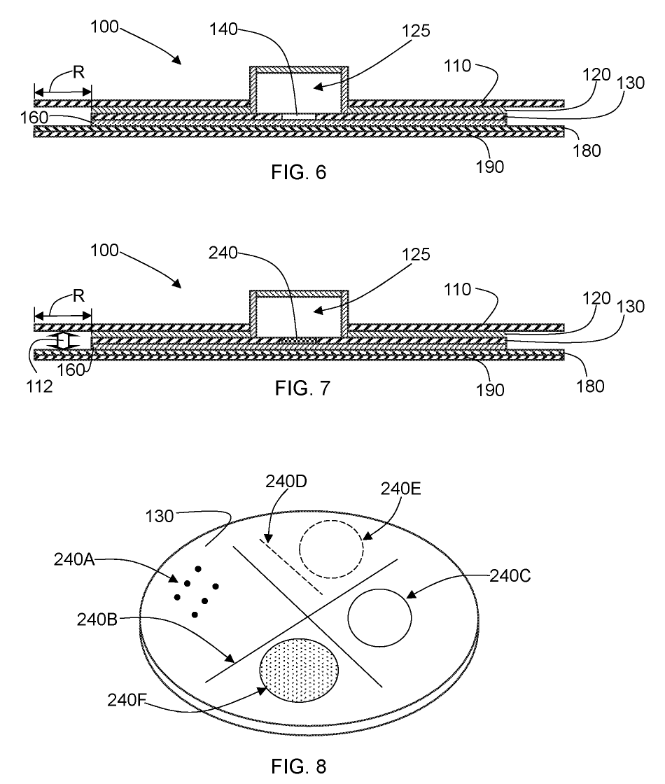

Referring now to FIG. 6, in an embodiment the second impermeable layer 130 has one or more openings 140 disposed therethrough (one is shown). In this embodiment, medicament in the form of a liquid, gel, cream, or powder contents 126 (see FIGS. 2, 9, and 10) stored within the reservoir 125 is allowed to exit through the one or more openings 140 to impinge on the impregnable substrate layer 160. Although FIG. 6 shows only one reservoir 125 with one opening 140, there can be two openings 140 each disposed over one of two reservoirs like the geometry shown in FIGS. 4 and 5. Further, in other embodiments, there can be three, four, five or more reservoirs 125, each reservoir 125 having an associated opening 140 on the second impermeable layer 130 over the reservoir 125. Regardless of the number of reservoirs 125, each reservoir 125 can contain the same or different contents 126 as described above and below.

As was noted above, the impregnable substrate layer 160 is coupled to the second impermeable layer 130 around at least a periphery of the impregnable substrate layer 160. There is therefore a continuous region of fixation 161 (see FIG. 2) around the impregnable substrate layer 160. Although FIG. 6 is drawn showing the impregnable substrate layer 160 laying flat against the second impermeable layer 130, the region of fixation 161, in fact, creates the pocket 162, as shown in FIGS. 2 and 5, between the impregnable substrate layer 160 and the second impermeable layer 130. The contents 126 of the reservoir or reservoirs 125 can flow into the pocket 162 and disburse laterally along the impregnable substrate layer 160, which helps in mixing of multiple different contents 126. The lateral disbursement into the pocket also facilitates the contents 126 being evenly absorbed into the impregnable substrate layer 160 to more evenly and fully permeate the impregnable substrate layer 160.

Because the embodiment of FIG. 6 does not include a pull tab 150 or other material directly covering the one or more openings 140, the contents 126 that have been absorbed into the impregnable substrate layer 160 could seep through the impregnable substrate layer 160. To prevent this from happening, the peel-off backing layer 170 of the prior embodiments is replaced with a third impermeable layer 180 of material. The third impermeable layer 180 is coupled to the impregnable substrate layer 160 at least around a perimeter of the impregnable substrate layer 160. The third impermeable layer 180 can be a peel-off layer.

The third impermeable layer 180 keeps the contents 126 that have seeped through the impregnable substrate layer 160 contained. In an embodiment, the third impermeable layer 180 is a label or packaging layer having printing or other information on it. In another embodiment, the third impermeable layer is not a label, but an additional printable layer of material 190 is coupled over the third impermeable layer 180. The additional printable layer of material is a label having contents or other information on it. .

Referring now to FIG. 7, in an embodiment the second impermeable layer 130 has one or more frangible openings 240 disposed therethrough (one frangible segment is shown). In this embodiment there is a frangible opening 240 associated with each of one or more reservoirs 125. Although FIG. 7 shows only one reservoir 125 with one frangible opening 240, there can be two frangible openings 240 each disposed over one of two reservoirs like the geometry shown in FIGS. 4 and 5. Further, in other embodiments, there can be three, four, five or more reservoirs 125, each reservoir 125 having an associated frangible opening 240 on the second impermeable layer 130 over the reservoir 125. Regardless of the number of reservoirs 125, each reservoir 125 can contain the same or different contents 126 as described above and below.

Still referring to FIG. 7, in this embodiment, each of the one or more frangible openings 240 is initially closed but can be burst open in response to increased pressure in the reservoir portion 125 over which the one or more frangible openings 240 are located. Upon the bursting or breaking open of the or more frangible openings 240, contents 126 of the reservoir 125 (or each of the reservoirs 125) in the form of a liquid, gel, cream, or powder is allowed to exit through the one or more frangible openings 240 to impinge on the impregnable substrate layer 160.

As was noted above, the impregnable substrate layer 160 is coupled to the second impermeable layer 130 around at least a periphery of the impregnable substrate layer 160. There is therefore a continuous region of fixation 161 (see FIG. 2) around the impregnable substrate layer 160. Although FIG. 7 is drawn showing the impregnable substrate layer 160 laying flat against the second impermeable layer 130, the region of fixation 161, in fact, creates the pocket 162, as shown in FIGS. 2 and 5, between the impregnable substrate layer 160 and the second impermeable layer 130. The contents 126 of the reservoir(s) 125 can flow into the pocket 162 and disburse laterally along the impregnable substrate layer 160, which helps in mixing and being evenly absorbed into and permeating the impregnable substrate layer 160. Regardless of the number of reservoirs 125, each reservoir 125 can contain the same or different contents 126 as described above and below.

Similar the embodiment of FIG. 6, the embodiment of FIG. 7 does not include a pull tab 150 and instead includes a frangible opening 240 that could undesirably burst before being used by a user. Such an undesirable burst event could cause the contents 126 to seep through the impregnable substrate layer 160. To prevent this from happening, the peel-off backing layer 170 of the prior embodiments is replaced with the third impermeable layer 180 of material as described for FIG. 6. In another embodiment, as was mentioned above, an additional printable layer of material 190 is coupled over the third impermeable layer 180. The additional printable layer of material is a label having contents or other information on it.

Referring to FIG. 8, several examples of frangible openings 240 are shown schematically. For example without limitation, the frangible opening 240 can be a point or points of weakness 240A, a line or lines of weakness 240B, any geometric shape of weakness 240C, a partial perforation arranged as a line 240D or any geometric shape 240E, or a thinned region of material 240F having any shape. Regardless of the particular implementation of the frangible openings 240A-240F, each can be burst open by an increased pressure within the reservoir 125 over which the frangible opening 240A-240F is positioned.

Although the wound care dressing pad 100 is illustrated herein as being generally circular, it can be any shape as desired or required to cover a wound, for example without limitation, round, square, oval, elliptical, triangular, rectangular, pentagonal, hexagonal, or any other regular or irregular polygonal shape. The wound care dressing pad 100 can have a longer dimension and a shorter dimension, for example, if it is rectangular or elliptical in shape. The wound care dressing pad 100 can be any size as desired or required to cover a wound, for example without limitation, having a shorter dimension or a longer dimension of any size from about less than an inch to about ten inches or more. Any two of the layers of the wound care dressing pad 100 disclosed herein as coupled to one another can be coupled by a sonic weld, a melt weld, an adhesive, or as otherwise known.

Materials for use as the first layer 110 include, for example without limitation, a cotton or polyester woven fabric that is flexible and breathable, a non-woven synthetic fabric that may be stronger and more durable, an elastic fabric that is advantageous for joint movement, a polyurethane (PU) film that is waterproof, especially thin, and transparent, polyethylene (PE), polyethylene terephthalate (PET), polyvinyl chloride (PVC), or silicone based films that are cost-effective and flexible, a hydrocolloid layer that promoted moist wound healing, or a foam-backed layer that provides cushioning and can be useful for blisters. The first layer 110 can also be made from a perforated polyurethane film that allows oxygen exchange or a microporous Teflon that repels water but allows vapor passage.

Adhesives for use on the first layer 110 to adhere the first layer to skin include, for example without limitation, acrylic based adhesives that are commonly available and provide strong adhesion, silicone adhesives that are gentle and reusable, hydrocolloid adhesives that are good for moist-healing and absorbing exudate, and zinc-oxide adhesive that is good for sensitive skin and long wear.

Materials for use as the impregnable substrate layer 160 include, for example without limitation, a non-woven fabric such as polypropylene, polyester that are commonly used in medical wipes, or a rayon-polyester blend that is soft and highly absorbent, a woven cloth such as a cotton gauze that is natural and breathable, bamboo fabric that is antibacterial and sustainable, or a nylon mesh that is strong and flexible, or a synthetic mesh with apertures such as an apertured polyester film that permits solution passage or a polyurethane foam with micro holes that facilitates a controlled release.

In some embodiments the impregnable substrate layer 160 is impregnated with a dry medicament or with a liquid medicament allowed to permeate into the impregnable substrate layer 160 and dry. In some embodiments the impregnable substrate layer 160 impregnated with either or both of the dry medicament or the liquid medicament permeated into the impregnable substrate layer 160 and dried is allowed to mix with a solution in the reservoir 125 at the time of use.

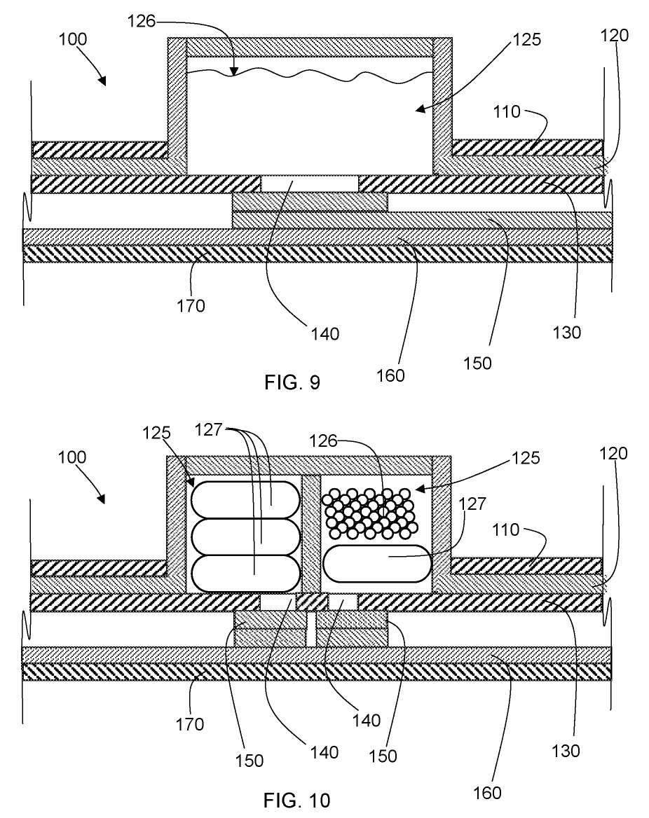

Referring to FIG. 9, as noted above, each of the one or more reservoirs 125 is configured to contain, hold, or store contents 126 that comprises a liquid, a gel, a cream, a powder or combinations thereof. In some embodiments, the liquid, gel, or cream contents 126 (shown as a liquid, gel, or cream 126 in FIG. 9) are loosely held within the reservoir 125 or reservoirs 125 such that the contents 126 are free to flow out form the reservoir 125 when the one or more openings 140 in this embodiment, or the one or more frangible openings 240 in other embodiments lacking pull tabs 150, provides a path for exit. Regardless of the number of reservoirs 125, each reservoir 125 can contain the same or different contents 126 as described above and below. Any of the embodiments disclosed herein having a pull tab 150 covering an opening 140 can also be produced using a frangible opening 240 without a pull tab 150, and with or without a third impermeable layer 180 as described above.

Referring to FIG. 10, in some embodiments, the reservoir 125 contains one or more capsules 127. Each of the one or more capsules 127 can include a liquid or a cream or a gel or a powder or any combination of a mix of a liquid or a cream or a gel or a powder. The capsules 127 can be burstable with a slight elevated pressure, for example by a user’s fingers, or may be dissolvable upon contact with other contents 126 or over time. Further, FIG. 10 shows an embodiment having two reservoirs 125, the reservoir 125 on the left holding three capsules 127 and the reservoir 125 on the right holding both a capsule 127 and a powder contents 126. In other embodiments, the same reservoir 125 can store or hold any combination or mix of capsules 127 and contents 126 comprising of a liquid, gel, cream, powder, or any combination thereof. Regardless of the number of reservoirs 125, each reservoir 125 can contain the same or different contents 126 or capsules 127 as described above.

Materials for use as the first impermeable film layer 120 that is formed into the reservoir 125 include, for example without limitation, thermoplastic polyurethane (TPU) that is durable and flexible, PE, polypropylene (PP), or PET that are cost-effective and medical grade, or polyvinyl alcohol (PVA). In addition, in some embodiments, the reservoir 125 can include a foam material within it for a gradual liquid release from the reservoir 125. In another embodiment, the first impermeable layer 120 can be made from a paper based film that is biodegradable. Materials for use in the second impermeable layer 130 and the third impermeable layer 180 include, for example without limitation, a medical grade film that prevents contamination. In some embodiments the second impermeable layer 130 is covered with a hydrophilic coating that improves solution absorption. Materials for the peel-off backing layer 170 and the additional printable layer 190 include, for example without limitation, a medical grade film that prevents contamination.

Material thicknesses for any of the layers described herein, including without limitation the first layer 110, the first impermeable film layer 120, the second impermeable film layer 130, the pull tab(s) 150, the impregnable substrate layer 160, the peel-off backing layer 170, the third impermeable layer 180, and the additional printable layer of material 190 have a material thickness between about 2 mil and about 12 mil. In some embodiments, one or more of the layers above (110, 120, 130, 150, 160, 170, 180, 190) can have different thicknesses from the other layers and in some other embodiments, one or more of the layers above (110, 120, 130, 150, 160, 170, 180, 190) can have a thickness less than 2 mils or greater than 12 mils.

Considerations for selections of the material layers described above include usage environment, and size and severity of the wound, Additional selections include without limitation, skin sensitivity, sterility and longevity, flexibility and comfort, and biodegradability. For example, skin sensitivity can require the use of hypoallergenic adhesives for medical use. Sterility and longevity can require materials that prevent contamination while ensuring solution stability. Flexibility and comfort are especially important when the wound care dressing pad 100 is being applied to joints. Biodegradability can be a consideration when desiring an eco-friendly material option for sustainability.

The wound care dressing pad 100 as disclosed provides a moist environment for wound healing, which promotes the growth of healthy cells, and facilitates the process of healing. The pull tab(s) 150 can prevent any contents of the reservoir(s) 125 from leaking prior to use. The pull tab(s) 150 can also allow a user to select which of several reservoirs 125 they would like to access for use, or to allow the selective mixing of two or more of the reservoirs 125. In the embodiments without the pull tab(s) 150, the frangible openings 240 similarly allow the selection of one or more of the reservoirs 125 to be accessed. The peel-off backing layer 170 is removably coupled to the first film layer 110 to cover and protect the impregnable substrate layer 160 until the wound care dressing pad 100 is ready for use.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

What is claimed is:

1. A wound care dressing pad, comprising:
a first film layer;
a first impermeable film layer formed to include at least one reservoir, the first impermeable layer coupled to the first film layer such that the at least one reservoir extends through the first film layer, the first film layer extending beyond a periphery of the first impermeable layer;
a second impermeable film layer coupled to the first impermeable film layer around at least a periphery of the at least one reservoir to enclose the at least one reservoir, the second impermeable film layer having an opening disposed therethrough and aligned with each of the at least one reservoirs of the first impermeable film layer;
an impregnable substrate layer coupled to the second impermeable layer around at least a periphery of the impregnable substrate layer; and
a peel-off backing layer removably coupled to the first film layer.

2. The wound care dressing pad of claim 1, wherein
the second impermeable film layer has one opening disposed therethrough; and
a pull tab is removably coupled to the second impermeable film layer to cover the one opening, wherein a portion of the pull tab extends beyond the first film layer.

3. The wound care dressing pad of claim 1, wherein
the first impermeable film layer is formed to include two reservoirs;
the second impermeable film layer has two openings disposed therethrough, each of the openings aligned with one of the two reservoirs; and
a pull tab is removably coupled to the second impermeable film layer to cover each of the two openings, wherein a portion of each of the pull tabs extends beyond the first film layer.

4. The wound care dressing pad of claim 3, wherein each of the two reservoirs contains a different medicament.

5. The wound care dressing pad of claim 4, wherein at least one of the medicaments is a powder.

6. The wound care dressing pad of claim 1, wherein
the first impermeable film layer is formed to include two reservoirs;
the second impermeable film layer has two openings disposed therethrough, each of the openings aligned with one of the two reservoirs; and
a single pull tab is removably coupled to the second impermeable film layer to cover both of the two openings, wherein a portion of the single pull tab extends beyond the first film layer.

7. The wound care dressing pad of claim 6, wherein each of the two reservoirs contains a different medicament.

8. The wound care dressing pad of claim 7, wherein at least one of the medicaments is a powder.

9. The wound care dressing pad of claim 1, wherein the peel-off backing layer a printable label.

10. The wound care dressing pad of claim 1, wherein the second impermeable film layer is covered with a hydrophilic coating.

11. The wound care dressing pad of claim 1, wherein the first impermeable film layer and the second impermeable film layer each have a thickness in a range between about 2 mil and about 12 mil.

12. A wound care dressing pad, comprising:
a first film layer;
a first impermeable film layer formed to include at least one reservoir, the first impermeable layer coupled to the first film layer such that the at least one reservoir extends through the first film layer, the first film layer extending beyond a periphery of the first impermeable layer;
a second impermeable film layer coupled to the first impermeable film layer around at least a periphery of the at least one reservoir to enclose the at least one reservoir, the second impermeable film layer having a frangible opening aligned with each of the at least one reservoirs of the first impermeable film layer;
an impregnable substrate layer coupled to the second impermeable layer around at least a periphery of the impregnable substrate layer; and
a third impermeable film layer coupled to the impregnable substrate layer around at least a periphery of the impregnable substrate layer.

13. The wound care dressing pad of claim 12, wherein
the first impermeable film layer is formed to include two reservoirs;
the second impermeable film layer has two frangible openings disposed therethrough, each of the frangible openings aligned with one of the two reservoirs.

14. The wound care dressing pad of claim 13, wherein each of the two reservoirs contains a different medicament.

15. The wound care dressing pad of claim 14, wherein at least one of the medicaments is a powder.

16. The wound care dressing pad of claim 12, wherein the third impermeable film layer is a printable label.

17. The wound care dressing pad of claim 12, wherein the first impermeable film layer and the second impermeable film layer each have a thickness in a range between about 2 mil and about 12 mil.

18. The wound care dressing pad of claim 12, wherein the frangible opening aligned with each of the at least one reservoirs of the first impermeable film layer is a line of weakness.

19. The wound care dressing pad of claim 12, wherein the frangible opening aligned with each of the at least one reservoirs of the first impermeable film layer is a partial perforation arranged as a line.

20. The wound care dressing pad of claim 12, wherein the frangible opening aligned with each of the at least one reservoirs of the first impermeable film layer is a partial perforation arranged as a circle.
